(19) United States
(12) Patent Application Publication
(10) Pub. No.: US 2021/0206665 A1
(43) Pub. Date: Jul. 8, 2021

(54) Light Sanitization

(71) Applicant: Lightcraft Technologies, LLC, Manassas, VA (US)

(72) Inventors: Rohullah Latif, Fullerton, CA (US); Jacob Daniel Conner, Rogersville, MO (US); Michael Keith Blankenship, Bristow, VA (US)

(73) Assignee: Lightcraft Technologies, Manassas, VA (US)

(21) Appl. No.: 17/209,114

(22) Filed: Mar. 22, 2021

Related U.S. Application Data
(60) Provisional application No. 63/046,660, filed on Jun. 30, 2020.

(51) Int. Cl. C02F 1/32; A61L 2/24; A61L 2/10

(52) U.S. Cl. CPC C02F 1/325; A61L 2/24; A61L 2/10; A61L 2202/11; C02F 2307/06; C02F 2303/04; C02F 2201/3228; C02F 2301/022

(57) ABSTRACT

A light sanitization system and method is disclosed that provides neutralization of pathogens and chemicals by embedding light waveforms within a stream of fluid.

### RELATED APPLICATIONS

The present application claims priority under U.S. 35 U.S.C. § 120 from U.S. provisional patent application 63/046,660 titled Light Sanitization filed on Jun. 30, 2020.

### FIELD OF THE INVENTION

The present invention relates to the field of sanitization and more specifically to the field of plumbing fixtures.

### BACKGROUND

There is a simple knowledge that has traveled with mankind for most of his recorded history: sunlight cleanses. This result was understood well before the explanation was clear. The legendary humanitarian Florence Nightingale observed that sunlight helped heal wounded soldiers and insisted that hospitals be constructed to allow the free entry of sunlight. In many instances, the proponents of light and light derivatives were the subject of some ridicule. It was believed in Tudor England that hanging red curtains aided patients recovering from the plague. In much the same way that advocates of willow bark to cure headaches were marginalized, so too were the proponents of red curtains in windows to ameliorate the plague. Observational trial-and-error outpaced science mankind until the last two centuries when mankind would learn about molecular interactions and the constituents of light to explain that, yes, acatecylic acid (i.e., ASPIRIN) from willow bark successfully treats headaches, and that light can be filtered into constituents to create red/infrared therapy treatments.

In addition to mankind's advances in the physical science, so too have the advances in the biological sciences explained that viruses and bacteria are evolving in an ever-increasing capacity to resist antibiotics. Bacteria are evolving metabolic and other workarounds to treatments calculated to kill the bacteria. Nothing, however, is immune to energy—in sufficient doses.

Therefore, there is a need for a sanitization process that is effective, inexpensive, widely-usable, and relatively quick.

### SUMMARY

The present invention is directed to a fluid sanitization system for an object. Currently the state of the art in “washing” an object is to place the object under running fluid, e.g., water to initially rinse away debris while using soap or other chemical to eradicate unwanted pathogens and chemical agents. The present invention is a leap forward in the art of washing; namely transforming water into the pathogen-killing element. The fluid is turned into a carrier for pathogen-killing photons that are trapped within the bounds of the fluid until contact with the object.

The present invention includes a fluid reservoir composed solely of a substantially-pure fluid. The present invention is capable of use with any fluid, particularly water, that has a relatively high refractive index with respect to the refractive index of the atmosphere in which the fluid will be released. Water has such a substantially high refractive index with respect to the majority of the Earth's atmosphere. The invention includes a faucet with an outlet, a housing, and conduit. The outlet directly accesses the exterior environment having the atmosphere, and for purposes of the present invention, the atmosphere with a refractive index substantially lower than the fluid refractive index. The faucet housing includes a faucet channel having a fluid transformer adapted to align fluid molecules into a laminar fluid stream throughout a cross-section of said fluid. This laminar flow is maintained from the channel to the faucet outlet. This laminar flow provides the basis for entrapping photons within the bounds of the fluid. Furthermore, there is faucet conduit that conducts fluid from said reservoir into said housing.

A photon emission source is positioned to emit into the fluid's laminar fluid stream a photon stream composed of photons having a waveform selected to a provide at least an LD50 dose. LD50 is usually time-determinative, and for purposes of the present invention, the time can be considered relative to the time that a reasonable user would position an object under the faucet for its purposes, e.g. shower faucets vs. sink faucets. The LD50 can be considered on a timescale equivalent to the coverage of the object with the fluid. For example, for hands, seconds, or for an automobile, minutes.

A method of the present invention includes channeling fluid that is then arranged and maintained in an orientation until release. Prior to release a photon emission stream is emitted into the newly oriented fluid. Substantially-pure fluid is channeled from a reservoir into a faucet channel, the fluid having a substantially higher refractive index than the surrounding atmosphere. The faucet channel has an outlet directly exposed the atmosphere. The fluid flow is molecularly arranged throughout a cross-section within the channel into a laminar flow orientation. This orientation is maintained from until it reaches the outlet. A photon emission stream, having a photon waveform, is into said laminar flow orientation prior to said outlet such that said photon emission bombardment is substantially contained within said fluid subsequent to escape via the. The waveform is adapted to provide at least an LD50 dose to a predetermined pathogen or agent.

These aspects of the invention are not meant to be exclusive. Furthermore, some features may apply to certain versions of the invention, but not others. Other features, aspects, and advantages of the present invention will be readily apparent to those of ordinary skill in the art when read in conjunction with the following description, and accompanying drawings.

### BRIEF DESCRIPTION OF THE DRAWINGS

FIG. 1A is a side, plan view of the system of the present invention.

FIG. 1B is a side, plan revealed view of the system of the present invention.

FIG. 1C is a side, exposed view of the system of the present invention.

FIG. 1D is a perspective, exposed view of the system of the present invention.

FIG. 2A is a perspective view of the system of the present invention.

FIG. 2B is a side, plan revealed view of the system of the present invention.

FIG. 2C is a perspective, exploded view of the system of the present invention.

FIG. 3A is a side, plan revealed view of the system of the present invention.

FIG. 3B is a perspective, exposed view of the system of the present invention.

FIG. 4A is a top, plan view of the transformer of the present invention.

FIG. 4B is a perspective of the transformer of the present invention.

FIG. 4C is a partially exploded view of a transformer of the present invention.

FIG. 5 is a view of method of the present invention.

FIG. 6 is a view of method of the present invention.

### DETAILED DESCRIPTION

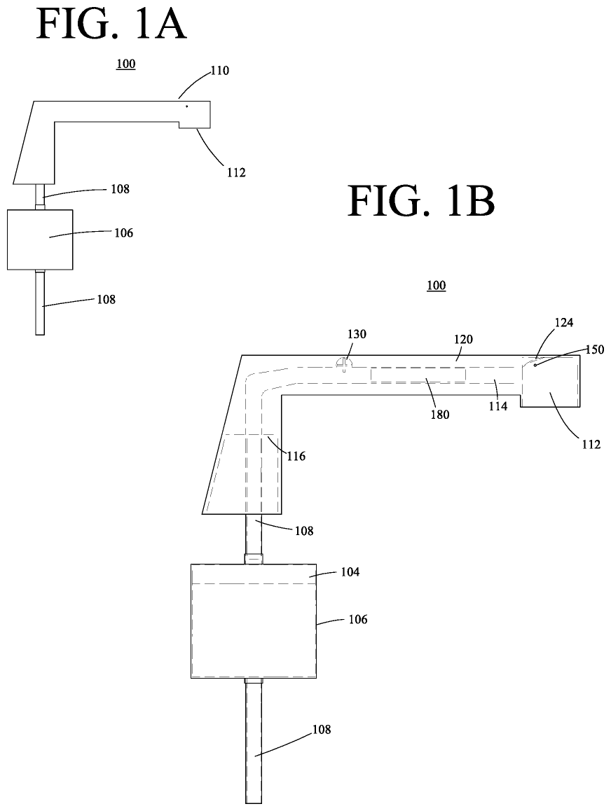

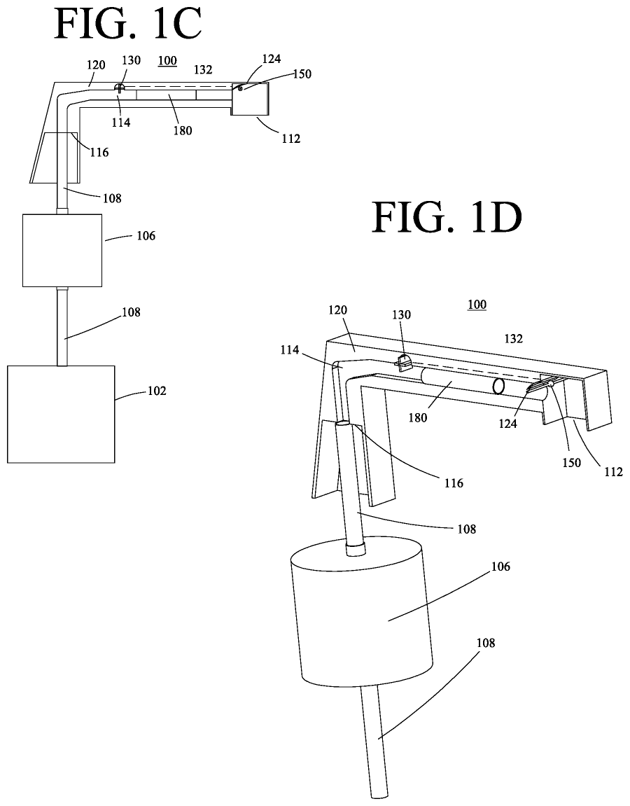

Referring first to FIG. 1, a basic embodiment of the system 100 of the present invention is shown. The system includes acts upon fluid. The ideal fluid is water because of its universality and exceptional cleansing qualities. Because of water's near-universality, water will often be discussed as the fluid of the present invention, but anywhere that water is discussed, this can be considered exemplary and simply as a placeholder for “fluid,” unless the properties of another fluid include attributes that prevent it from being used with the present invention.

Fluid 900 is made available to the present invention 100. The system is meant to be made available irrespective of fluid, and fluid will be discussed purely to the extent that it is acted upon by the present invention in the same way that a hammer is distinct from nails, however, it is often helpful to describe a hammer relative to a nail. Water in the state that it is made available to persons personally and commercially is often clean, but far from pure. Artesian wells, for example, often have significant deposits of sulfur within the water made available therefrom, and water delivered by a utility company often includes minerals and fluoride. Water, and other fluid “as delivered” can be considered as original fluid 904 for purposes of the present disclosure; and although such fluid may be capable of use with the present invention, it is preferred that fluid be modified to be a in substantially-pure state. Substantially pure fluid 900, for purposes of the present invention includes a liquid constituted principally only of a single type of molecule to the extent that additional molecules do not alter the capacity of the fluid to carry one or more photon streams (as will be discussed later).

There can multiple types of fluids are substantially pure for the present invention. An example of a substantially pure fluid is water having been filtered for fluoride and otherwise de-ionized. Water deionized to the standards of a rudimentary chemistry laboratory is a prime example of substantially pure fluid. Deionized water is made by running tap water, spring water, or distilled water through an electrically charged resin. Usually, a mixed ion exchange bed with both positive and negative charged resins is used. Cations and anions in the water exchange with H+ and OH− in the resins, producing H2O (water). Distilled water may be used with the present invention. Distilled water is a type of demineralized water that is purified using the process of distillation to remove salts and particulates. Usually, the source water is boiled and the steam is collected and condensed to yield distilled water.

The present invention can use more than a single molecule if mixed to be a substantially intermixed such that the properties of the fluid can be maintained during transit throughout the faucet 110 of the present invention. For example, glacial acetic acid or vinegar can be used with the present invention because the mixture includes a stability that will remain substantially constant throughout the faucet notwithstanding that glacial acetate includes less than one percent water, “anhydrous (water-free) acetic acid” and vinegar often includes from three to nine percent acetic acid. So long as the water is substantially free of extraneous molecules, and the acetate is substantially free of non-acetate molecules, the combination is substantially pure for purposes of the present invention. Fluids that may suitable candidates for use with the present invention include: Water; Ethanol; Methanol; N-propanal; Butanol; Ether; Dichloromethane; Carbon disulfide; glycerol; Acetone; Carbon tetrachloride; Cyclohexane; Formic acid; Toluene; Anisole; Pyridine; Acetic Acid; Hexane; Xylene; Trifluoroacetic acid; Dimethyl sulfoxide; benzene; Nitrobenzene; Quinoline; Dibutyl phthalate; Dimethylformamide; Cyclohexane; Anisole; Tetrahydrofuran; Petroleum ether; etc. It is worth mentioning again that although a solvent relative to one atmosphere may not be suitable candidate, use of the present invention in a different atmosphere 902 may make such previously non-suitable candidate worthwhile.

The base level of substantially pure is filtering the fluid to remove as much extraneous materials as is commercially practicable while maintaining a suitable stream of fluid. A filter on the grade used for personal-use refrigerators creates “substantially pure” water for purposes of the present invention because it transforms the original fluid to a state more receptive to that of the present invention. Accordingly, at its most basic level, the creation of substantially pure fluid can be any reasonable activity sought to increase the amenability of fluid to increase the refractive index of the fluid with respect to the atmosphere into which the fluid will be released.

The original fluid 904 passes through one or more filters 104 in a cartridge to perform any of the purification discussed above or otherwise is in accord with the present invention. Fluid is delivered 210 via fluid conduit 108 to, and perhaps into, the faucet 110 of the present invention. By faucet is its meant such articles that accept moving fluid and exhaust moving fluid within a single housing 120. The fluid 900 is accepted within the housing 120 into a faucet channel 114. The faucet channel 114 for purposes of the present invention is simply such pathway within the faucet that allows the transmission of fluid therethrough. In conventional sink faucets, for example, water is delivered through conduit 108 until well into the body where there is a faucet seal wall 116. Here, conduit 108 may cease and the internal surfaces 122 act as the water pathway. Whether there is a distinct pipe, or simply void, such pathway is the channel 114 for purposes of the present invention.

Thus far, in a conventional faucet, water would be chaotically or tortuously be proceeding through the plumbing fixture. Such water, or fluid, is must now be molecularly rearranged 220 into a laminar flow orientation. Laminar flow for purposes of the present invention is meant to be considered as known to a person of ordinary skill in the art. Laminar flow is flow for a type of fluid (gas or liquid) flow in which the fluid travels smoothly or in regular paths, in contrast to turbulent flow. Turbulent flow is flow wherein the fluid undergoes irregular fluctuations and undertakes conflicting vectors. Laminar flow is often referred to as streamline flow, in other words: the velocity, pressure, and other flow properties at each point in the fluid remain constant. As explained by the Encyclopedia Britannica” “Laminar flow over a horizontal surface may be thought of as consisting of thin layers, or laminae, all parallel to each other. The fluid in contact with the horizontal surface is stationary, but all the other layers slide over each other.” Laminar flow, Encyclopedia Britannica, (May 11, 2018) Encyclopedia Britannica, Inc.

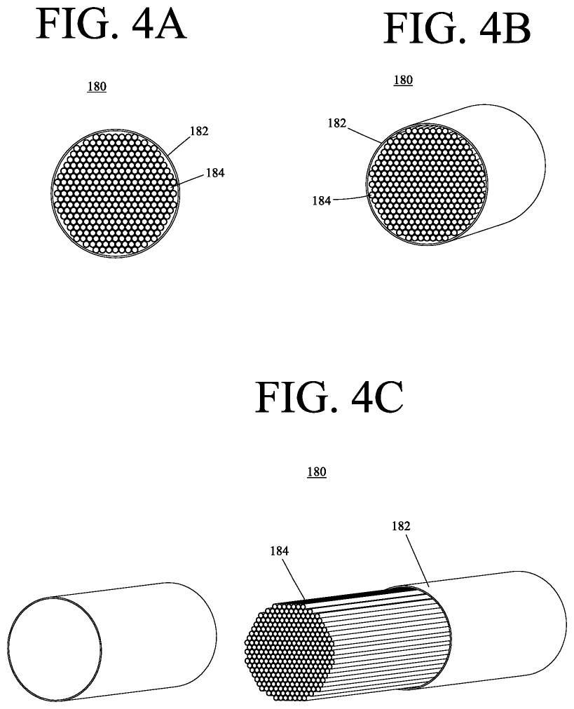

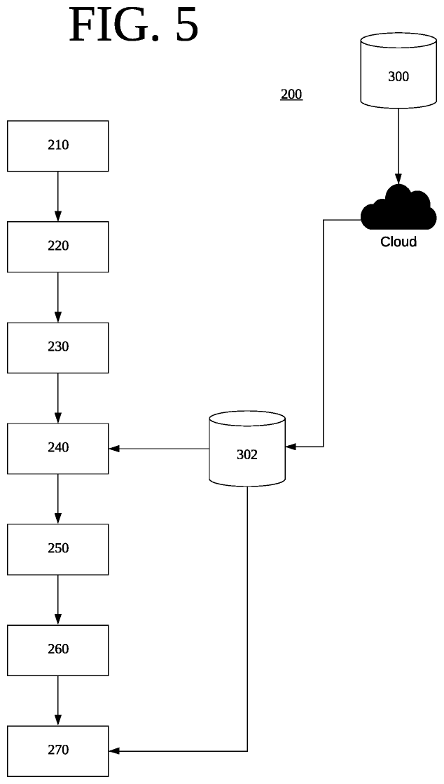

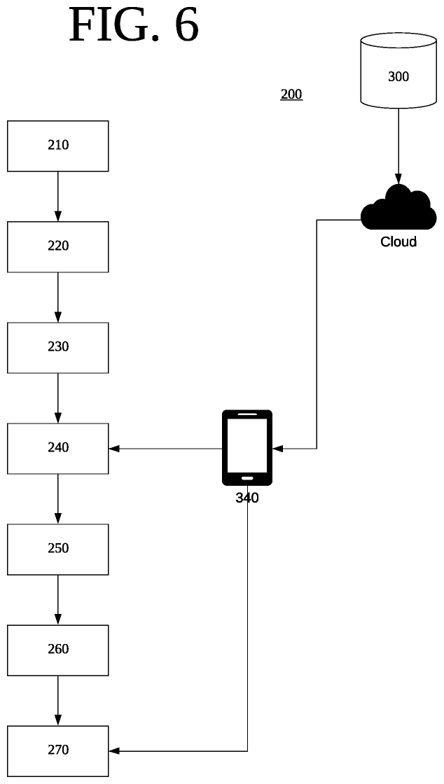

A transformer 180 accepts the turbulent flow of fluid from the channel 114 and rearranges the fluid into a laminar flow stream. As can be seen in greater detail in FIG. 4, an exemplary transformer includes a conduit with an outer sheath 182 that acts as a boundary 184 that supports a myriad of internal microconduit members 184. The finer the microconduit, the greater the stability the laminar flow will possess, excepting that a microconduit on a scale approaching the molecular size of the water molecule itself will cause unwanted collisions and impede the goals of laminar flow of the present invention. Turbulent water enters the entry point 180a of the transformer, is conducted in the micoconduit, and exits 180b the transformer.

Returning to FIG. 1, From the point of exit from the transformer 180 through the remainder of the faucet channel 144 until the point of exit of the faucet via the faucet outlet 112. The period can be termed throughout the present disclosure as the laminar flow stream, and while the water is maintained 230 in this laminar flow stream, the water (or other fluid) may be manipulated in advantageous ways for purposes of the present invention.

The creation of the laminar flow should be performed with adequate consideration. The entirety, that is to say the overwhelmingly majority, of the fluid in the channel must undertake this arrangement. Preferred versions of the present invention ensure that the entirety of the cross-section of the flow of fluid 900 passes through the channel 114. Leaks and permeable barriers in the transformer lead to degradation of the laminar flow stream and degradation of the result of the present invention.

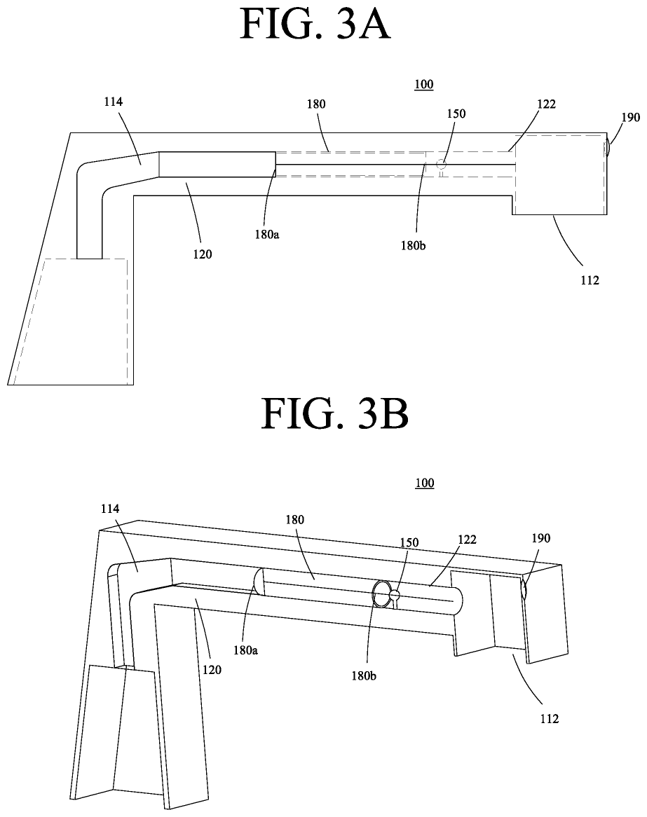

A photon emitter 150, or photon emission source, transmits photons into the laminar flow stream, and if performed suitably, the photon emission will stay within the stream of water—even upon exit. Essentially, and this is a rough analogy, water and other fluids will act as a sheath for the photons in the same manner that a fiber optic cable supports total internal reflection of photons, irrespective of path. The analogy breaks down readily because fluid is less stable than the fibers of fiber optic cables, and significant curves and other pathway interruptions create the turbulent environment that impedes laminar flow and internal reflectance. Accordingly, a key feature of the present invention is maintaining 230 a laminar flow such that turbulence is not returned to the fluid while in the faucet. Accordingly, the light should maintain as straight of a path as possible, and perpendicular paths should be avoided, if a curve is present in the faucet exists at all. Experimentation has shown that photons will remain embedded within the water cross-section notwithstanding curvature. Water, for example, will maintain substantial internal reflectance of Ultraviolet light for curvature approximately equal to the degree of curvature that water traveling parallel to the Earth's surface experiences as it lacks parallel support and falls toward the Earth's surface. In preferred inventions, curvature in the channel 114 subsequent to the transformer is equivalent or only slightly departs from this curvature. In other words, if one were to make a hole in the side of a container and allow water to drain therefrom, that curvature should be the curvature maintained by the faucet—or curvature similar. However, in preferred embodiments of the present invention downward curvature in the faucet channel 114 can be a key feature in supplementary maintenance of internal reflectance. For example, as seen in FIG. 3, the inner wall 122 of the housing 120 include internal reflectance cladding that ensures that the photons within the fluid maintain the maximum amount of internal reflectance upon exit via the faucet outlet 112.

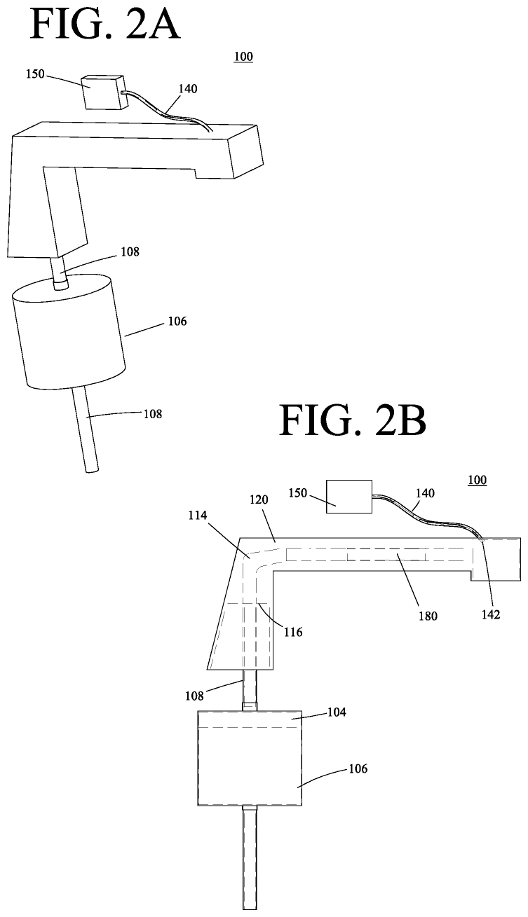

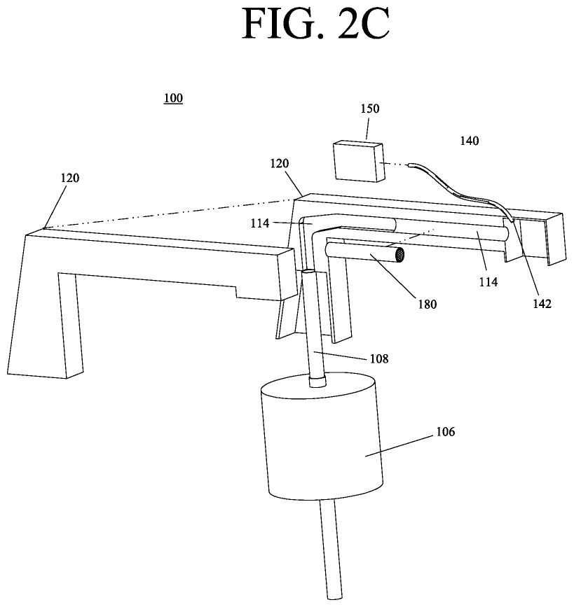

As shown in FIG. 1, the photon emitter 150 is itself placed within the faucet housing 120. The photon emitter 150 is within the laminar stream flow so as to embed 240 the photon emission stream into the fluid 900 rushing thereby. Naturally, the use of a physical object in the stream of the fluid 900 leads to laminar fidelity degradation. Accordingly, as can be seen in FIG. 2, the present invention includes a photon emission source 150 that is placed outside of the laminar flow stream, while nonetheless in the faucet housing 120, which then illuminates a reflective member 124 which then passes the light to the laminar flow stream. More preferred is the act of aligning the photon emission stream with the laminar flow stream. In the embodiment of FIG. 2, the emitter 150 is not merely placed adjacent to the reflective member, but at the focal point of the emitter such that light from the emitter 150 is reflecting in a regular array. If the liquid, having passed from the transformer, undertakes an orientation similar to that of the light, then the light and the fluid flow will be “in-phase” (for purposes of the present invention). Light “in-phase” in meant the arrangement of light travel and fluid flow travel such that the components are traveling in essentially parallel and co-mingling paths.

Once embedded, the light will be maintained within the protective barrier formed by the fluid until substantial disruption. Experimentation has shown that the light will maintain substantial fidelity within the water casing through a fall based on gravity and until contact with an object. In other words, water can be made to carry light energy, and for purposes of the present invention, the light used includes light waveforms known to be harmful to pathogens and chemical agents. The following pathogens and agents are known to be subject to destruction or neutralization based on energy bombardment:

See table.

Source ClorDiSys, Inc. Application Note #12, Ultraviolet Light Disinfection Data Sheet

The present invention is principally utilized with UV light in the UV-C range because it is shown to principally have all of the pathogen/agent destruction of other UV bands while not capable of penetrating skin. However, the present invention does not need to be utilized with UV light. Because the dosage period of the normal act of washing is short, and the act itself so important to human interaction, the energy level of light utilized with the present invention can include any intensity or wavelength suitable for use with targeted pathogens or agents.

LD50 for purposes of the present invention means the amount of energy or other factor needed to eliminate or denature 50% of a given population. Often LD50 is based on a calculation of time and amount/degree of form—and also based on living objects. Because the present invention can be utilized to kill pathogens or denature chemicals, that is to say, alter their chemical or physical structure such that they no longer maintain the attributes that make the cleaning beneficial, LD50 is used loosely. One cannot necessarily provide a “lethal dose” to digitalis; however, one can however apply energy to destabilize the molecule such that its toxic nature is altered. Other forms in which the invention can be expressed to the “nines” of denaturization. “To the first nine” is 90%+ efficacy in neutralizing a pathogen or chemical, to the “second nine” is 99%+ and to “the third nine” is 99.9%+ and so on. When combating pathogens a dose that minimally neutralizes pathogens is insufficient. Pathogens often multiple at an exponential rate that requires destruction well past the 50% mark that suits the standards in complex living organisms.

Furthermore, the present invention can used indications 190 provided by an indicator based on an agent or pathogen of concern. Because this subject matter is novel, and the idea that water itself is the cleansing agent, could be a new experience for a series of customers, the present invention could include an indicator that serves two or more purposes. The first purpose may to indicate that the light transmission, which preferably happens away from direct user visibility, is happening at all; but also, an indicator may secondly provide an indication of an appropriate length to wash one's hands based on the intensity and other characteristics of the light. For purposes of the present invention, emission and light “waveform” is meant to include those characteristics of the wave that are being guided into the fluid. Waveform characteristics may include intensity of the wave, the wavelength, frequency, and other identifying characteristics of the wave.

A waterwheel 150 may be placed in the pathway of the fluid as it passes through the channel. Preferably as a generator is a source of turbulence, the waterwheel 150 is placed prior to the transformer 180. It is preferred that the faucet of the present invention not necessitate direct connection to a household electrical circuit, particularly since water pressure in a typical American suburban fixture system is sufficient to turn a waterwheel that can utilize gearing to turn a generator that can directly connect to the emitter 150, or connect to a form of energy storage either capacitor or battery for long term powering options. In other embodiments, long term energy storage is utilized to permit the present invention to engage in transmission reception with computing devices to be able to indicate 270 to users the length of time sufficient to wash hands with energy-bearing-fluid. Furthermore, the indicator 190 may be a guide as to the activity or other operation of the faucet and emitter. In a preferred embodiment, the emitter only lights when the emitter is active. Light can be hard to visualize in the bright light common in bathrooms and other areas with plumbing. For the visually impaired, the indicator may be the only basis for knowing that the emitter is actively injecting light into the fluid. In other embodiments, the indicator may include multiple lighting wavelengths, e.g. blue, green, red, yellow, etc., to indicate any condition of power or operation, such as low battery, a particular waveform being utilized, blinking to indicate the near end of object washing, etc.

The faucet can have memory that includes timing for general washing, washing for specific pathogens/chemicals, or otherwise. The timing can be built-in or provided/updated post-hac.

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. A fluid sanitization system for an object comprising:
a fluid reservoir composed solely of a substantially-pure fluid with a fluid refractive index;
a faucet with (i) a faucet outlet directly accessible to an exterior environment having an atmosphere with a refractive index substantially lower than said fluid refractive index, (ii) a housing with a faucet channel having a fluid transformer adapted to align fluid molecules into a laminar fluid stream throughout a cross-section of said fluid maintained from said channel to said faucet outlet, (iii) a conduit, adapted to channel fluid from said reservoir into said housing; and
a photon emission source, positioned to emit into said laminar fluid stream a photon stream composed of photons having a waveform selected to a provide at least an Log1 dose to a predetermined pathogen on a timescale substantially equivalent to coverage of said object with said fluid.

2. The system of claim 1 wherein said faucet further comprises a generator accessible to said faucet channel and adapted to directly power said photon emission source.

3. The system of claim 1 wherein said photon emission source is positioned within said faucet channel.

4. The system of claim 3 wherein said system includes a reflective surface member wherein said channel.

5. The system of claim 4 wherein said faucet housing includes an interior surface bearing said reflective surface member as interior surface cladding.

6. The system of claim 1 wherein said photon emission source is positioned external to said faucet and includes a cable having internal reflection with a cable outlet positioned within said faucet channel to emit said photon stream having into said laminar fluid stream.

7. The system of claim 1 further comprising an indicator adapted to alter a visible state based on operation of said photon emission source.

8. The system of claim 1 further comprising a filter adapted to selectively omit fluid other than a predefined fluid.

9. A method of sanitization of an object within a given atmosphere, said method comprising:
channeling a substantially-pure fluid with a substantially higher refractive index than the atmosphere into a faucet with a faucet channel with an outlet directly exposed the atmosphere;
molecularly arranging said fluid flow throughout a cross-section of said fluid flow within said channel into a laminar flow orientation;
maintaining said laminar flow orientation of said fluid to said outlet; and
injecting via photon emission stream bombardment a photon waveform into said laminar flow orientation prior to said outlet such that said photon emission bombardment is substantially contained within said fluid subsequent to escape via said outlet, said waveform adapted to provide at least an LD50 dose to a predetermined pathogen on a timescale substantially equivalent to coverage of the object with said fluid.

10. The method of claim 9 further comprising the step of energizing said photon emission stream with a generator contacting said substantially pure fluid prior to laminar flow orientation arrangement.

11. The method of claim 9 wherein said injecting step includes injecting via a photon emission source positioned within said faucet to contact said laminar flow orientation of said fluid therewith.

12. The method of claim 9 wherein said injecting step includes guiding said photon emission stream from a photon emission source external to said faucet to a photon stream outlet positioned within said laminar flow orientation of said fluid.

13. The method of claim 9 wherein said injecting step includes injecting via a photon emission source positioned adjacent to a reflective member adapted to directly orient said photon emission stream within said laminar flow orientation of said fluid.

14. The method of claim 13 wherein said injecting step includes injecting via a photon emission source positioned adjacent to a reflective inner surface of said faucet adapted to directly orient said photon emission stream within said laminar flow orientation of said fluid.

15. The method of claim 9 further comprising filtering an original fluid to form said substantially pure fluid.

16. A method of sanitization of an object within a given atmosphere, said method comprising:
channeling a substantially-pure fluid with a substantially higher refractive index than the atmosphere from a reservoir into a faucet with a faucet channel with an outlet directly exposed the atmosphere;
molecularly arranging said fluid flow throughout a cross-section of said fluid flow within said channel into a laminar flow orientation;
maintaining said laminar flow orientation of said fluid to said outlet;
injecting via photon emission stream bombardment a photon waveform into said laminar flow orientation prior to said outlet such that said photon emission bombardment is substantially contained within said fluid subsequent to escape via said outlet, said waveform adapted to provide at least an LD50 dose to a predetermined agent for a coverage timescale; and
providing a sensual indication proximate to said faucet upon completion of said coverage timescale.

17. The method of claim 16 further comprising transmitting a coverage timescale over a network from a master Agent Information Database to an Arithmetic Logic Unit (“ALU”) in communication with persistent storage in signaled communication with said faucet to alter said coverage timescale based on a desired agent.

18. The method of claim 17 further comprising transmitting characteristics of said waveform over a network from a master Agent Information Database to an Arithmetic Logic Unit (“ALU”) in communication with persistent storage in signaled communication with said faucet to alter said waveform based on a desired agent.
